(19) United States
(12) Patent Application Publication
(10) Pub. No.: US 2003/0171755 A1
(43) Pub. Date: Sep. 11, 2003

(54) Bone screws

(76) Inventors: Colin F. Moseley, Los Angeles, CA (US); Jan Koreska, Glendale, CA (US)

Correspondence Address: FISH & RICHARDSON PC, 225 FRANKLIN ST, BOSTON, MA, 02110, US

(21) Appl. No.: 10/092,204

(22) Filed: Mar. 5, 2002

(51) Int. Cl. A61B 17/58

(52) U.S. Cl. 606/73

(57) ABSTRACT

The invention relates to a spinal fixation system containing a rod positioned contiguous to and spanning a length of the spine. The rod is held in place by a bone screw that has a double offset at the proximal end, which is connected to a clamping mechanism. The bone screw is secured to the bone and the rod is secured in the clamping mechanism, whereby the clamping mechanism of the new double offset bone screw can be easily moved to accommodate the location of the rod.

### TECHNICAL FIELD

This invention relates to segmental spinal instrumentation systems, and more particularly to pedicle screws for such systems.

### BACKGROUND

In the last two decades, surgeons have moved toward systems that provide a secure grasp of individual vertebrae, and that enable intricate correction of complex spinal deformities. These systems are called segmental spinal instrumentation systems, because they can secure each segment (vertebra) of the spine.

Such segmental systems include three main components, rods, hooks, and bone screws. The hooks are used to attach to the arches of the vertebrae, and come in several sizes to accommodate various sizes of vertebrae. The rods are long and thin, but strong enough to be fairly rigid. The bone screws are screwed directly into the vertebrae from the posterior aspect, or in some aspects are screwed into spaces between vertebrae. They are also called “pedicle screws” because they are typically inserted into the “pedicle” of the vertebrae. Bone screws come in a variety of shapes and sizes.

Both the hooks and the screws are connected or clamped to the rods by various setscrews, clamps, nuts, collars, wedges, or brackets, to rigidly secure them to the rods.

### SUMMARY

The invention is based on the discovery that if the shaft of a bone screw is bent or offset to form an S-curve, the resulting screw can be used more effectively to secure a rigid rod to multiple vertebrae.

In general, the invention features a bone screw that includes a screw shaft having a double offset or double bend at a proximal end; a head fixed to the shaft proximal to the double offset; and a clamping mechanism rotatably secured to the head. The central axes of the head and the screw shaft can be parallel or angled. The screw shaft can be threaded from the distal end to a distalmost offset of the double offset, or only part way up the screw shaft. The clamping mechanism can further include a fixation member, such as a setscrew, and the clamping mechanism can be a U-shaped body that defines a channel for receiving a rod. In the new bone screw, the head can be an integral part of the shaft distal to the double offset, or it can be a separate part that is fixed to the shaft.

In another aspect, the invention features a spinal fixation system that includes a rod to be positioned contiguous to and spanning a length of the spine along multiple vertebrae; and a plurality of the new bone screws. Of course, such systems can also include various hooks, and the bone screws can be in a variety of sizes. The double offset of these bone screws can comprise two right-angle bends. In this case, the bone screw comprises a shaft having a first central axis and a proximal end having a second central axis, and the first and second central axes are parallel. In other embodiments, the bends are not quite right angles, and then these axes can be angled with respect to each other. In all embodiments, the screw shaft of these bone screws can rotate independently of the clamping mechanism.

In some embodiments, the head of the bone screw includes a protrusion configured to contact the rod in use. This protrusion keeps the rod from sliding within the clamping mechanism after the rod is secured into the clamping mechanism. When the rod is secured, the screw shaft and the clamping mechanism are fixed at a selected angle. In the new systems, the clamping mechanism can comprise a U-shaped body that defines a channel for receiving the rod, and the base of the clamping mechanism can have an aperture through which a proximal end of the bone screw protrudes. In some embodiments, the channel is open at the top to allow insertion of the rod into the channel, and the arms of the U-shaped body have female threading which contact male threading on the fixation member to secure the rod.

In another aspect, the invention also features a method for aligning a spinal column by obtaining two or more of the new bone screws; securing a first bone screw into a first vertebra; securing a second bone screw into a second vertebra; positioning a rod along the spinal column; rotating the bone screw shafts and clamping mechanisms to accommodate the rod and bring the vertebra into proper alignment; and securing the rod to each clamping mechanism. For example, the rod can be secured to the clamping mechanisms with setscrews.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a schematic three-dimensional view of a new spinal fixation system.

FIG. 2 is an oblique view of a new bone screw.

FIG. 3 is a side view of a new bone screw.

FIG. 4 is a three-dimensional view of a new spinal fixation system oriented and engaged to a vertebra.

Like reference symbols in the various drawings indicate like elements.

### DETAILED DESCRIPTION

FIG. 1 shows the general elements of a spinal fixation system 10 in accordance with the present invention. The system includes a bone screw 12 configured to connect to a bone, e.g., a vertebra, a rod 14 that sets a bone structure, e.g., a spinal column, in a fixed position, and a fixation member 16 that clamps the rod 14 to the bone screw 12. In a preferred embodiment, at least two or more bone screws 12 are used with one rod 14.

In particular, the bone screw 12 of the spinal fixation system 10 contains a clamping mechanism 20 at the proximal end 37 of the screw shaft 30. The clamping mechanism 20 has the ability to rotate 360°, rock left-to right, and rock forward-to-backward on an enlarged head 32 on the proximal end 37 of the screw shaft 30. The clamping mechanism 20 includes a U-shaped body 21 with two arms 22a and 22b forming a channel 24 designed to receive the rod 14. The channel 24 ends in an edge on opposite sides of the U-shaped body 21.

In one embodiment, the channel 24 is open, halfway up the diameter and throughout the length of the channel 24 to receive the rod 14. In another embodiment, the channel 24 is enclosed with openings at one or both ends for insertion of the rod 14. An enclosed channel 24 will have an opening on the surface enabling communication with a fixation member 16.

In one embodiment, the rod 14 has a diameter that is slightly smaller than the inner dimensions of channel 24. Therefore, for a snug fit, a sleeve may be inserted into the channel 24 that is configured to fit in the channel 24 and to accept the dimensions of the rod 14. On the surface of the arms 22a and 22b facing the channel are female threads 26 that mate with male threads 18 on the fixation member 16. The clamping mechanism is immobilized to the screw shaft 30 when the rod 14 is secured to the clamping mechanism 20.

The clamping mechanism 20 is connected to the proximal end 37 of the screw shaft 30 in an opening in the clamping mechanism 20. The clamping mechanism 20 contains an aperture through a wall of the clamping mechanism where the distal end 38 of the screw shaft 30 extends or protrudes from the aperture. The aperture narrows or has a recess or restriction that is smaller than the head 32 and stops the screw shaft 30 from proceeding through the aperture. The screw shaft 30 retains the ability to rotate with respect to the clamping mechanism 20, enabling the clamping mechanism 20 to be positioned into close proximity to the rod 14, even after the screw shaft 30 is set in bone.

In one embodiment, the clamping mechanism 20 has the form of a U-shaped body 21 with an open top. In other embodiments, the clamping mechanism 20 has a C-shaped body with an open side. The connection remains dependent on the presence of an aperture in the clamping mechanism 20 for the head 32 of the screw shaft 30 to engage the clamping mechanism 20.

The head 32 of the screw shaft 30 can be attached to the clamping mechanism 20 in a variety of ways. In one embodiment, the head 32 of the screw shaft 30 is spherical and fits into the clamping mechanism 20 in a manner that allows for a “ball-and-socket” motion. This type of connection allows for the greatest range of mobility for the clamping mechanism 20 to swivel on the head 32 of the screw shaft 30. In another embodiment, the head 32 takes the form of a hemisphere to form a semi-“ball-and-socket” joint. The semi-“ball-and-socket” mechanism allows a lesser degree of mobility of the clamping mechanism 20 relative to the screw shaft 30, but may provide greater stability in some circumstances. A third embodiment includes a loosely fitted connection between the head 32 and the clamping mechanism 20, allowing for a more restricted degree of mobility, but complete rotation between the head 32 and the clamping mechanism 20 along the central axis of the screw shaft 30.

In a specific embodiment, the head 32 of the screw shaft 30 is restrained within the clamping mechanism 20 via congruent contact surfaces with shapes that are part of a sphere. This allows completely free rotation of the clamping mechanism 20 about the head of the screw, and tilting of the screw within the clamping mechanism 20 of about 15 to 45 degrees, e.g., 30 degrees, both laterally and longitudinally.

FIG. 1 shows grooves 28a and 28b in the arms 22a and 22b respectively, in the U-shaped body 21. In one embodiment, the grooves are recessed into arms 22a and 22b to a desired depth. In another embodiment, the grooves extend through the width of the clamping mechanism 20 and form apertures. Once the bone screw has been driven into the bone using a driving instrument, grooves 28a and 28b are engaged by a different instrument to position and hold the clamping mechanism 20 in place while guiding the rod 14 and the setscrew 16 into place.

The bone screw 12 contains a distal pointed end 38 for engaging a bone, and a proximal end 37 that are separated by a threaded shank 36 of the screw shaft 30 that is designed for securing the screw in the bone. The type of threading, the diameter, and the length of the threaded shank 36 can vary as required for different sizes and types of bones.

Proximal to the threaded shank 36 lies the double offset 34a and 34b adjacent the head 32. The first offset or bend 34a from the distal end 38 of the bone screw 12 is angled away from the axis of the bone screw 12. The second offset or bend 34b from the distal end 38 of the bone screw 12 angles back in the direction of, e.g., to become parallel to, the axis of the bone screw 12. The double offset enables the clamping mechanism 20 to cover a greater circumference when the screw shaft 30 is rotated relative to the circumference generated if the screw shaft 30 was straight. The greater circumference allows a greater opportunity for positioning the clamping mechanism 20 with respect to the rod 14.

The rod 14 has a generally uniform cylindrical cross-section and is manufactured from a medically inert substance, e.g., a metal such as titanium or stainless steel. Other materials that have the same characteristics as titanium or steel may also be used. The rod 14 is configured to fit into the channel 24 of the clamping mechanism 20.

The fixation member 16 has male threads 18 on its outer surface that mate with the female threads 26 on the inner surface of the U-shaped body 21. The fixation member 16 can be a setscrew and can have a plurality of socket configurations, e.g., hexagonal or octagonal. The fixation member is inserted into the U-shaped body of the clamping mechanism 20 by using a driving instrument, for example, a screwdriver or a wrench. For example, the fixation member 16 can be configured with a hexalobe shaped, e.g., Torx® socket, and turned with a hexalobe shaped, e.g., Torx® driver, or other conventional sockets.

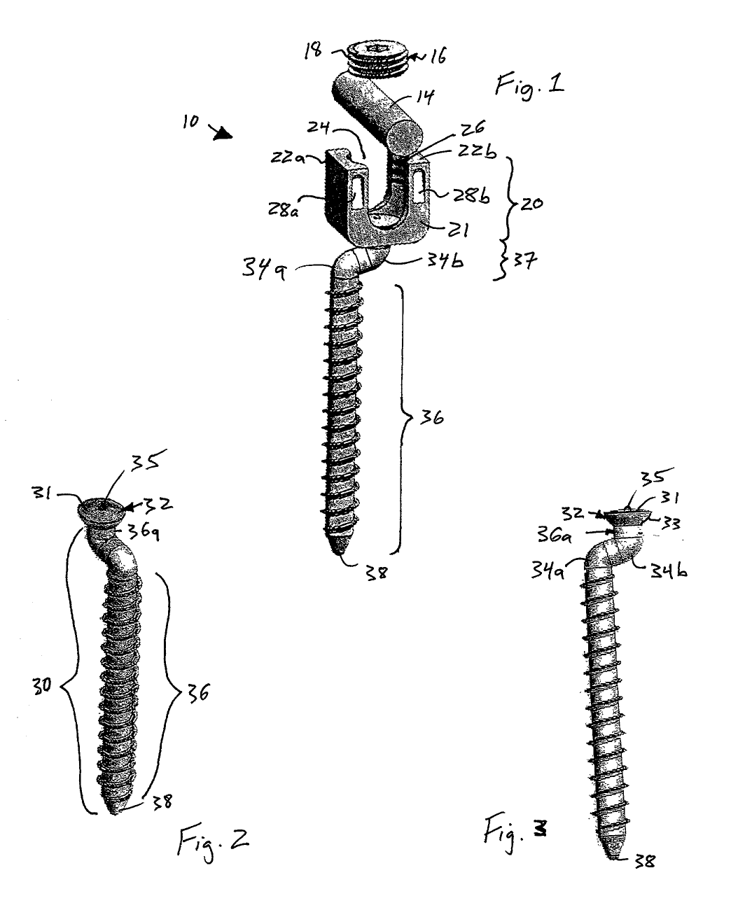

FIG. 2 is an oblique view of the screw shaft 30 absent the clamping mechanism. The pointed distal end 38 is separated from the proximal end 37 by the threaded shank 36. The threaded shank 36 is followed proximally by the offsets 34a and 34b. The screw shaft 30 is completed at the proximal end 37 by the enlarged head 32. Preferably, the head 32 will have a partial spherical bottom 33 and a flattened or conical upper surface 31. This configuration allows for significant mobility of the screw shaft 30 relative to the clamping mechanism 20. The head 32 can, in some embodiments, have a recess for receiving an engaging tool. In most embodiments, a specially designed driving instrument wraps around the S-shaped double offset of the screw shaft 30.

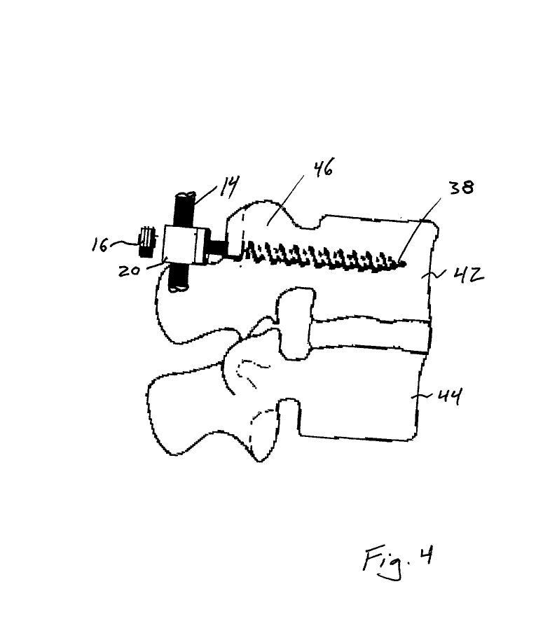

FIG. 3 is an orthogonal view of the screw shaft 30. It shows another angle to view the head 32 and the first and second offset 34a and 34b. In FIG. 3, the screw shaft 30 has a small projection 35 on the conical surface 31 on the top of the head 32. In one embodiment, this projection 35 contacts the rod 14 when the rod 14 is placed into the U-shaped body 21 of the clamping mechanism 20. Upon securing the rod 14 with the fixation member 16, the compression of the rod 14 onto the projection 35 secures the head 32 into the aperture of the clamping mechanism 20, keeps the rod from sliding laterally within the U-shaped body 21, and prevents angular changes in the shaft position relative to the U-shaped body 21.

Referring again to FIG. 3, the double offset 34a and 34b is visible as an S-shaped curve. In another embodiment, the offsets 34a and 34b can be at different angles to each other and to the screw shaft 36. For example, as seen in FIG. 3, the central axes of a small section 36a of the shaft adjacent the head 32 (which coincides with the central axis of this head 32) and the main threaded shaft 36 are parallel to each other. However, by varying the angle, the central axes of the small section 36a and the threaded shank 36, one can adjust the reach of the clamping mechanism 20 as it rotates about the main shaft 36. By increasing the angle, the reach is increased, and decreasing the angle, the reach is decreased.

FIG. 4 illustrates a method of using the spinal fixation system. A first vertebra 42 is to be fixed to a second vertebra 44. The distal end 38 of the bone screw 12 is driven into the first vertebra at the pedicle region 46 to a predetermined depth using a driving instrument. A second bone screw is driven into the second vertebra 44 in a similar fashion. Rod 14 is placed into the channel 24 of the clamping members 20, which are rotated to accommodate the rod. Once the rod is inserted into each clamping mechanism, the fixation members 16 are tightened into their respective clamping mechanisms 20, thereby securing the spinal fixation system. The rod 14 is secured to at least two bone screws 12 that are engaged on different vertebrae.

The spinal fixation system is manufactured and machined by standard techniques well known in the art, e.g., molding, milling, and threading. The materials used are medically approved and biologically inert. Such materials can include metals, e.g., titanium or steel. See, e.g., U.S. Pat. Nos. 5,797,911, 6,083,227, and 6,187,005.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A bone screw comprising:
a screw shaft comprising a double offset at a proximal end;
a head fixed to the shaft proximal to the double offset; and
a clamping mechanism rotatably secured to the head.

2. The bone screw of claim 1, wherein central axes of the head and the screw shaft are parallel.

3. The bone screw of claim 1, wherein central axes of the head and the screw shaft are angled.

4. The bone screw of claim 1, wherein the screw shaft is threaded from the distal end to a distalmost offset of the double offset.

5. The bone screw of claim 1, wherein the clamping mechanism further comprises a fixation member.

6. The bone screw of claim 1, wherein the fixation member is a setscrew.

7. The bone screw of claim 1, wherein the clamping mechanism comprises a U-shaped body that defines a channel for receiving a rod.

8. The bone screw of claim 1, wherein the head is enlarged.

9. The bone screw of claim 1, wherein the head is an integral part of the shaft distal to the double offset.

10. A spinal fixation system, comprising:
a rod to be positioned contiguous to and spanning a length of the spine; and
a plurality of bone screws of claim 1.

11. The system of claim 10, wherein the double offset comprises two right angle bends.

12. The system of claim 10, wherein the bone screw comprises a shaft having a first central axis and a proximal end having a second central axis, and wherein the first and second central axes are parallel.

13. The system of claim 10, wherein the clamping mechanism further comprises a fixation member.

14. The system of claim 10, wherein the screw shaft can rotate independently of the clamping mechanism.

15. The system of claim 13, wherein the fixation member is a setscrew.

16. The system of claim 10, wherein the head comprises a protrusion configured to contact the rod in use.

17. The system of claim 10, wherein securing the rod to the clamping mechanism fixes the screw shaft and the clamping mechanism at a selected angle.

18. The system of claim 10, wherein the clamping mechanism comprises a U-shaped body that defines a channel for receiving the rod.

19. The system of claim 10, wherein a base of the clamping mechanism comprises an aperture through which a proximal end of the bone screw protrudes.

20. The system of claim 18, wherein the channel is open on the top to allow insertion of the rod into the channel.

21. The system of claim 18, wherein the arms of the U-shaped body comprise female threading which contact male threading on the fixation member to secure the rod.

22. A method for aligning a spinal column, the method comprising:
obtaining two or more bone screws of claim 1;
securing a first bone screw into a first vertebra;
securing a second bone screw into a second vertebra;
positioning a rod along the spinal column;
rotating the bone screw shafts and clamping mechanisms to accommodate the rod and bring the vertebra into proper alignment;
and securing the rod to each clamping mechanism.

23. The method of claim 22, wherein the rod is secured to the clamping mechanisms with setscrews.
